(19) United States
(12) Patent Application Publication
(10) Pub. No.: US 2005/0010237 A1
(43) Pub. Date: Jan. 13, 2005

(54) Catheter to cannulate coronary sinus branches

(76) Inventor: Imran K. Niazi, River Hills, WI (US)

Correspondence Address: Philip G. Meyers Law Office, Suite 302, 1009 Long Prairie Road, Flower Mound, TX, 75022, US

(21) Appl. No.: 10/864,913

(22) Filed: Jun. 9, 2004

Related U.S. Application Data
(60) Provisional application No. 60/477,094, filed on Jun. 9, 2003.

(51) Int. Cl. A61B 19/00

(52) U.S. Cl. 606/129

(57) ABSTRACT

A catheter system suitable for introduction of a pacing lead into a branch of a human coronary sinus includes an outer, resilient catheter having shape memory which is configured for introduction into the coronary sinus from the right atrium of a human heart. The catheter has at least one preformed bend defining an acute included angle proximate a tip thereof, such that the catheter has a hooked end portion when in an undistorted condition. A stiff obturator is configured for sliding into and out of the catheter. A distal end portion of the obturator has a substantially straight configuration whereby introduction of the obturator fully into the catheter straightens the hooked end portion, and withdrawal of the obturator causes the hooked end portion to resume the hooked shape.

This application claims priority of U.S. Provisional Application No. 60/477,094, filed Jun. 9, 2003.

### FIELD OF THE INVENTION

This invention relates to medical devices, particularly to catheters of the type used to cannulate the coronary sinus and branches thereof.

### BACKGROUND OF THE INVENTION

Congestive heart failure is one of the commonest diagnoses leading to hospital admission in the United States. There are 2 to 5 million patients diagnosed with congestive heart failure in the U.S. annually, and 15 million worldwide. Treatment of heart failure consists of medications, and cardiac transplantation in severe cases. Other forms of surgery, e.g. valve surgery, are also sometimes helpful. Attention has recently focused on resynchronization therapy. About 30-50% of people with severe congestive heart failure have asynchronous contraction of the cardiac chambers. This can be corrected by pacing the right atrium, the right ventricle and the left ventricle at optimal intervals to provide synchrony. Leads to pace the right atrium and right ventricle have been used for years.

The coronary sinus is a venous structure that is three to four centimeters in length and one centimeter in diameter. It forms a part of the venous drainage of the heart. The coronary sinus arises from the posterior inferior aspect of the right atrium and courses over the posterior surface of the heart, ending in the great cardiac vein. It is the final common venous drainage of most of the heart.

Pacing the left ventricular epicardium via a coronary sinus branch for cardiac resynchronization is well established in theory. Catheter systems have been devised to allow delivery of a pacing lead to the main coronary sinus from where it is manipulated into a side branch. At the present time, introducing such a lead into any of the branches is considered sufficient; however, with increasing knowledge it is becoming apparent that a specific branch of the coronary sinus should be targeted for each patient to achieve optimum results.

Niazi U.S. Pat. No. 6,638,268, issued Oct. 28, 2003, describes a double catheter including an outer, resilient catheter having shape memory and a hook-shaped distal end, and an inner, pliable catheter slidably disposed in the outer catheter and of greater length than the outer catheter so that a distal end portion of the inner catheter can be extended or retracted from a distal end opening of the outer catheter to vary the overall length of the double catheter. The inner catheter preferably has an internal lumen suitable for the introduction of contrast media. A mechanism operable from the proximal end of the outer catheter is provided for changing the curvature of the hook shaped distal end of the outer catheter. Such a catheter can cannulate the coronary sinus without significant manipulation. This system permits the surgeon to vary the curvature of the catheter system, but does not address all of the problems encountered when attempting to cannulate the coronary sinus. In particular, a double catheter system significantly increases the size of the catheter, making it more difficult to manipulate in the coronary sinus and its branches.

Cannulating a selected branch of the coronary sinus to position a lead in a selected branch of the coronary sinus presents a number of challenges. For example, if the selected branch is acutely angled from the coronary sinus, a stylet driven lead cannot be used. In such cases, it may also be difficult or impossible to advance a guide wire into the branch to guide a catheter. Some branches of the coronary sinus have valves adjacent to the origin of the branch which are difficult to negotiate. In these cases, cannulating the branch provides more support when the lead is introduced into the branch. The posterior branch, or middle cardiac vein is in many instances in an especially viable location for positioning a lead, however, cannulation of the posterior branch can be particularly difficult due to its proximity to the coronary ostium insofar as withdrawal during manipulation of the catheter can result in displacement of the catheter from the coronary sinus altogether.

### SUMMARY OF THE INVENTION

A catheter system according to the invention is suitable for introduction of a pacing lead into a branch of a human coronary sinus. Such a system includes a resilient catheter having shape memory which is configured for introduction into the coronary sinus from the right atrium of a human heart, the catheter having at least one preformed bend defining an acute included angle proximate a tip thereof, such that the catheter has a hooked end portion when in an undistorted condition. A stiff obturator is configured for sliding into and out of the catheter. A distal end portion of the obturator has a substantially straight configuration whereby introduction of the obturator fully into the catheter straightens the hooked end portion, and withdrawal of the obturator causes the hooked end portion to resume its hooked shape. According to a preferred form of the invention, suitable means is provided for extending a wire through and out of the catheter from a location above the hooked end in order to act as a guide. Such means may take a variety of forms, such as a valve formed in a side wall of the catheter though which a guide wire can pass, or a side channel formed in the wall of the catheter. The catheter is preferably substantially J-shaped in its undistorted condition and includes a substantially straight stem portion, a curved mid-portion that is thinner and less stiff than the stem portion, and the hooked end portion extending from the mid-portion, which is thinner and less stiff than the mid-portion.

The system preferably utilizes a set of obturators that serve different purposes. Two or more generally J-shaped obturators are provided with varying curvature depending on the location of the coronary sinus ostium in different patients. In addition, an obturator shaped for introduction of a guide wire through its end is also provided as part of the set to carry out the procedure described hereafter.

A catheter system according to a second aspect of the invention includes a resilient catheter having shape memory and which is configured for introduction into the coronary sinus from the right atrium of a human heart, the catheter preferably having at least one preformed bend rendering the catheter generally L- or J-shaped when in an undistorted condition, and a mechanism operable from the proximal end of the catheter for changing the curvature of the distal end of the catheter, including a cable anchored to the catheter at a point proximate the distal end of the catheter and at a point proximate the proximal end of the catheter. Shortening of the cable from the point proximate the proximal end of the catheter results in increased curvature of the hooked end portion. The catheter further has suitable means for extending a guide wire through and out of the catheter from a location above the distal end portion.

A catheter system according to a third aspect of the invention includes a resilient catheter as in the second embodiment. A first mechanism operable from the proximal end of the catheter is provided for changing the curvature of the distal end portion of the catheter, including a first cable anchored to the catheter at a point near the distal end of the catheter and at a point near the proximal end of the catheter. Shortening of the first cable from the point near the proximal end of the catheter results in increased curvature of the distal end portion. A second mechanism operable from the proximal end of the catheter is also provided for changing the curvature of the distal end portion of the catheter at a different location form the first mechanism. The second mechanism includes a second cable anchored to the catheter at a point proximate the distal end of the catheter different from the point at which the first cable is attached, and at a point near the proximal end of the catheter. Shortening of the second cable from the point near the proximal end of the catheter results in increased curvature of the distal end portion at a location different from the increased curvature resulting from shortening of the cable of the first mechanism. This can be used to make the catheter assume an S-shaped configuration as further described below.

According to a further aspect of the invention, the resilient catheter having a hooked end is pre-bent sideways to make it easier to insert into the coronary sinus. Preferably the catheter has at least two, first and second preformed bends that give the catheter a curved, hooked shape proximate a tip thereof, which first and second bends lie in a common plane. A third preformed bend is provided that defines an acute included angle, which third bend extends out of the common plane of the first and second bends. The first and second bends typically render the catheter substantially J-shaped when in an undistorted condition, and the third preformed bend causes the catheter to hook to the left when viewed from its proximal end. This permits the catheter to more readily enter the ostium of the coronary sinus. These and other aspects of the invention are discussed further in the detailed description which follows.

### BRIEF DESCRIPTION OF THE DRAWINGS

In the accompanying drawings, wherein like numeral denote like elements:

FIG. 1 is a side view of a first catheter suitable for use in the catheter system of the invention;

FIGS. 2A-2C are side views of three obturators suitable for use in the catheter system of the invention;

FIG. 3 is a partial, enlarged bottom view of the catheter of FIG. 1;

FIG. 4 is an alternative embodiment of the catheter of FIG. 1 having a side branch and a Touhy-Borst applicator attached;

FIG. 5 shows the catheter of FIG. 1 assembled with the obturator of FIG. 2A;

FIGS. 6A-6H are schematic diagrams illustrated a method of use of the catheter system according to the invention;

FIG. 7 is a side view of an alternative catheter system according to the invention;

FIG. 8 is a cross-sectional view taken along the line 7-7 in FIG. 7; and

FIG. 9 is a cross-sectional view comparable to FIG. 8 according to an alternative embodiment of the catheter;

FIG. 10 is a schematic diagram showing advance of the guide wire through the valve while still in the right atrium;

FIG. 11 is a side view of a catheter and guide wire provided with radioopaque markers according to the invention;

FIG. 12 is a bottom view of the valve shown in FIG. 11;

FIG. 13 is a side view of a catheter of the invention provided with a catheter cuff and holes for contrast media injection according to the invention;

FIG. 14 is a schematic diagram of a single deflecting catheter according to the invention;

FIG. 15 is a cross sectional view of the catheter shown in FIG. 14;

FIG. 16 is a side view of a dual curve deflecting catheter according to the invention;

FIG. 17 is a side view of the catheter of FIG. 16 in a deflected position;

FIG. 18 is a schematic diagram of the dual deflecting catheter according to the invention;

FIG. 19 is a side view of an alternative embodiment of a catheter according to the invention; and

FIG. 20 is a top end view of the catheter of FIG. 19.

### DESCRIPTION OF PREFERRED EMBODIMENTS

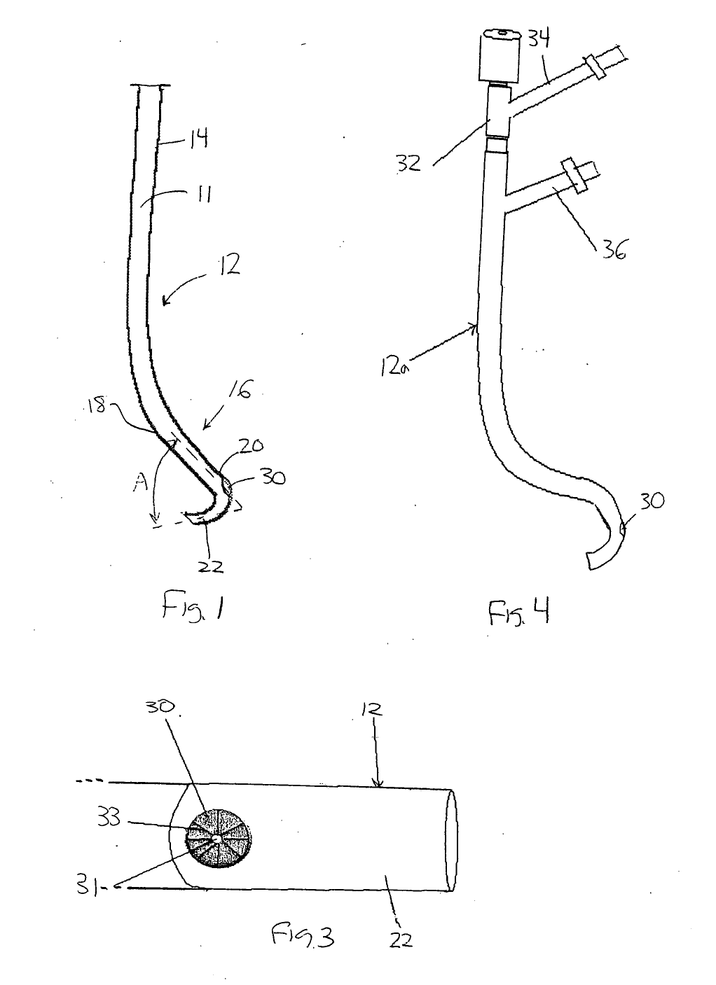

Turning now to FIGS. 1 to 5, a catheter system 10 according to the invention includes an obturator 40a and an outer, resilient catheter 12 having shape memory which is configured for introduction into the coronary sinus from the right atrium of a human heart. Catheter 12 is formed from a pliable material such as Silastic® silicone rubber that takes the shape of relatively stiff obturator 40a inserted into catheter 12. While pliable, catheter 12 has sufficient radial strength to prevent the catheter from collapsing when it is advanced and positioned across a bend or curve.

In FIG. 1, catheter 12, illustrated in a relaxed or undistorted state, includes a relatively straight stem segment 14 and a curved end 16 approximately 7 to 12 cm long. Curved end 16 is formed with a primary curved segment 18, a secondary curved segment 20 and a distal end segment or hooked end 22. Secondary curved segment 20 is formed with a bend or curve having an included angle A of from about 60° to about 120°. To facilitate cannulation of lateral branches of the coronary sinus that diverge at acute angles, A is preferably 90° or less. Distal end segment 22 extends from 0.5 to 1.5 cm beyond secondary curved segment 20, forming a hooked end. Secondary curved segment 20 is directed in substantially the opposite direction from first curved segment 18, giving catheter 12 an overall S-shape when it its undistorted state.

In order to facilitate use of the catheter 12 with J-shaped obturator 40a, catheter 12 is preferably formed with portions having varying degrees of stiffness. Thus, straight segment 14 is firm and relatively stiff, secondary segment 22 is more pliable than segment 14, and end segment 22 is softer and more highly pliable than secondary segment 22. The variable stiffness may be accomplished by constructing segments 20, 22 with correspondingly thinner walls than segments 14, 20 respectively, or by using a layered construction wherein catheter 12 is constructed with a first, relatively stiff layer and one or more softer layers that extend further than the thick layer to form segments 20, 22.

Catheter 12 is preferably provided with a flexible silicone valve 30 for passage of a guide wire 38, such as a 0.014″ to 0.035″ diameter steel wire, from the interior of catheter 12 into the main coronary sinus. Valve 30 is preferably located at or near the bend of secondary curved segment 20 where it meets distal end segment 22. As shown in FIG. 3, valve 30 is circular with a small central aperture 31 and radial cuts 33 in the silicone to allow passage of the guide wire 38. Radiopaque markers of the type described in FIG. 12 below may be placed around the outside of valve 30 and around aperture 31, respectively, to aid in locating valve 30 for purposes of threading wire 38 through it. Guide wire 38 extending through valve 30 is used as a rail to support catheter 12 as the catheter is manipulated during cannulation of a selected branch of the coronary sinus as explained hereafter. As exemplified in FIG. 4, catheter 12 may be fitted with a Touhy-Borst applicator 32 including a side branch 34. A further side branch 36 located below applicator 32 may be connected to IV tubing.

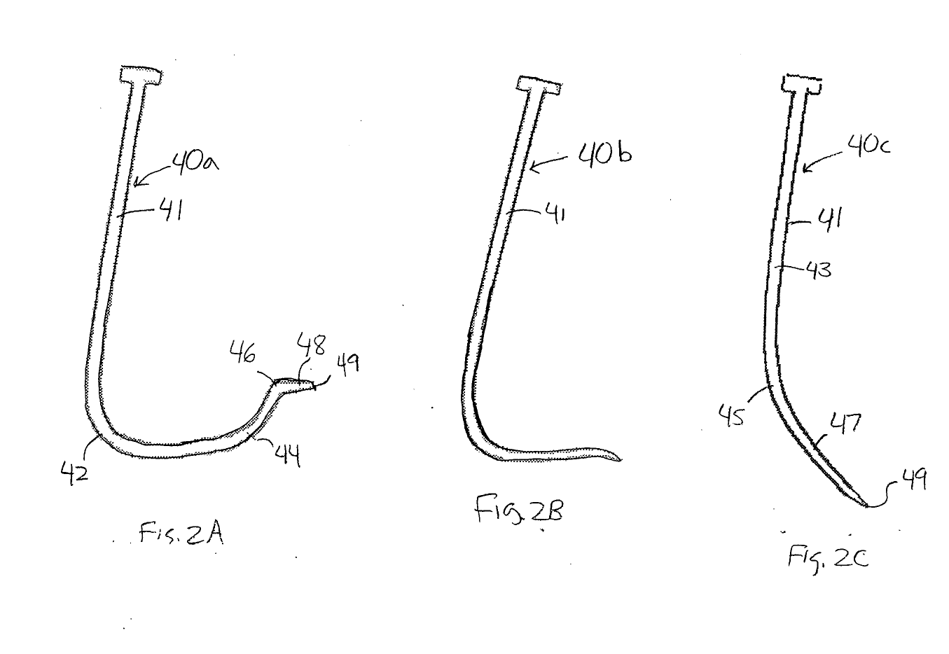

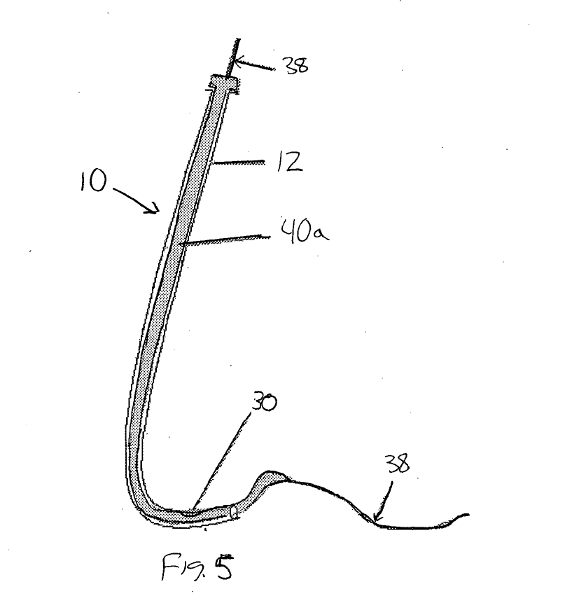

FIG. 2A through 2C illustrate obturators 40a-40c suitable for use with catheter 12. Obturators 40a-40c are shaped to generally conform to the stem portion of catheter 12 and are sufficiently stiff to cause the distal end segment 22 of catheter 12 to conform to the shape of the obturator when the obturator is inserted into lumen 11 of catheter 12. As illustrated in FIG. 2A, obturator 40a is J-shaped and includes a straight stem section 41, first, second and third curved segments 42, 44 and 46 and a short, straight distal end section 48. Obturator 40a is used with catheter 12 for cannulating the main coronary sinus, in particular when the coronary sinus ostium is located high in the right atrium, i.e., >1 cm. Alternatively, a more L-shaped obturator 40b is used with catheter 12 for cannulating the main coronary sinus when the origin of the coronary sinus is low. Obturator 40c has a single bend 45 that defines an obtuse angle (e.g., 130-160°) between a straight distal end portion 47 and stem 41. Obturator 40c has a lumen 43 ending in a small tip opening 49 that is used to pass a guide wire, typically 0.035 inch, through valve 30. Obturators 40a-40c may be coated with a hydrophilic lubricious material which allows the obturator to slide in and out of catheter 12 with ease.

FIG. 5 illustrates the catheter system 10 assembled from catheter 12 and obturator 40a with a guide wire 38 that passes through a tip opening 39 of obturator 40a. As illustrated, catheter 12 is distorted by the introduction of obturator 40a such that catheter 12 conforms to the shape of obturator 40a and is thus configured to cannulate the coronary sinus from the right atrium of a human heart.

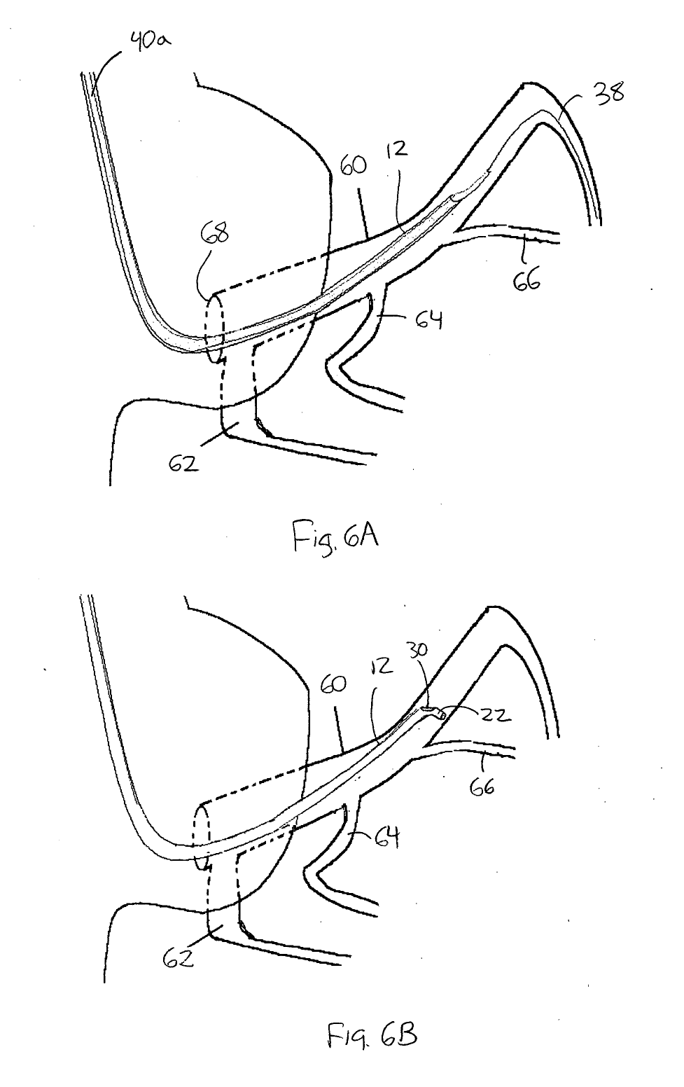

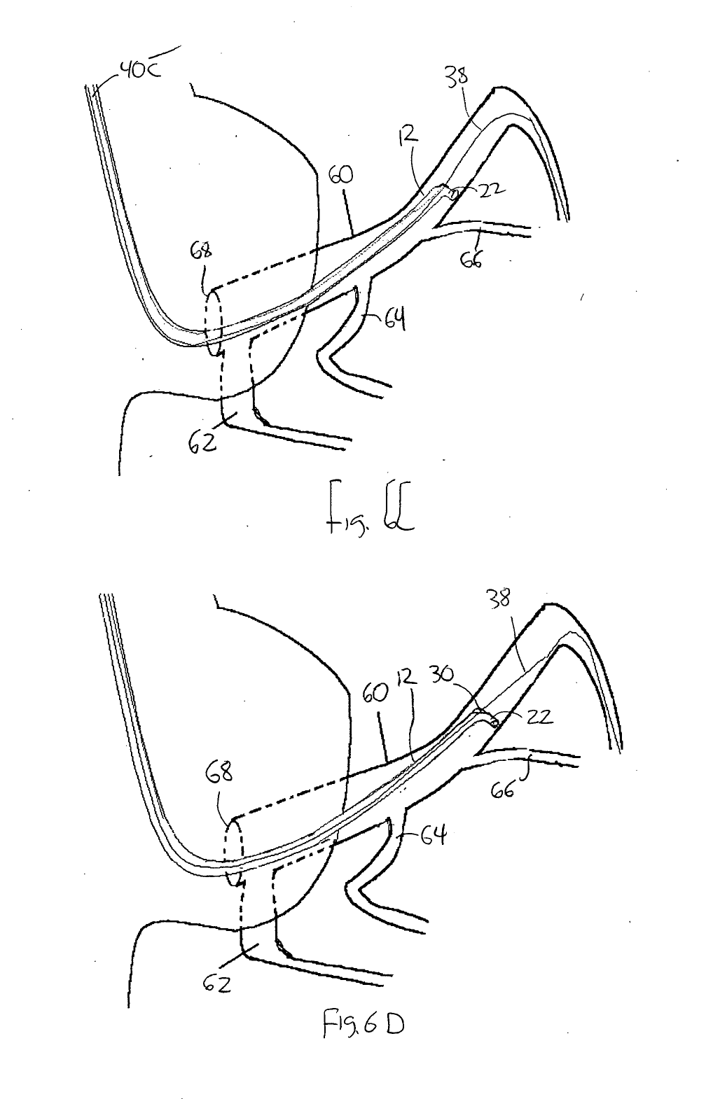

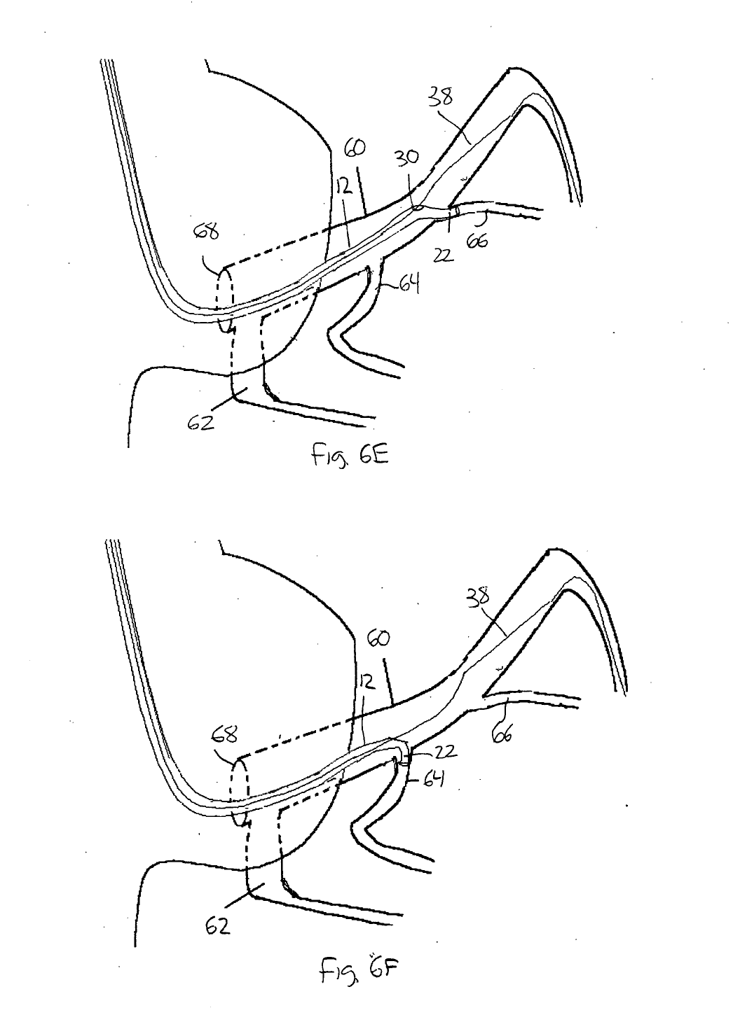

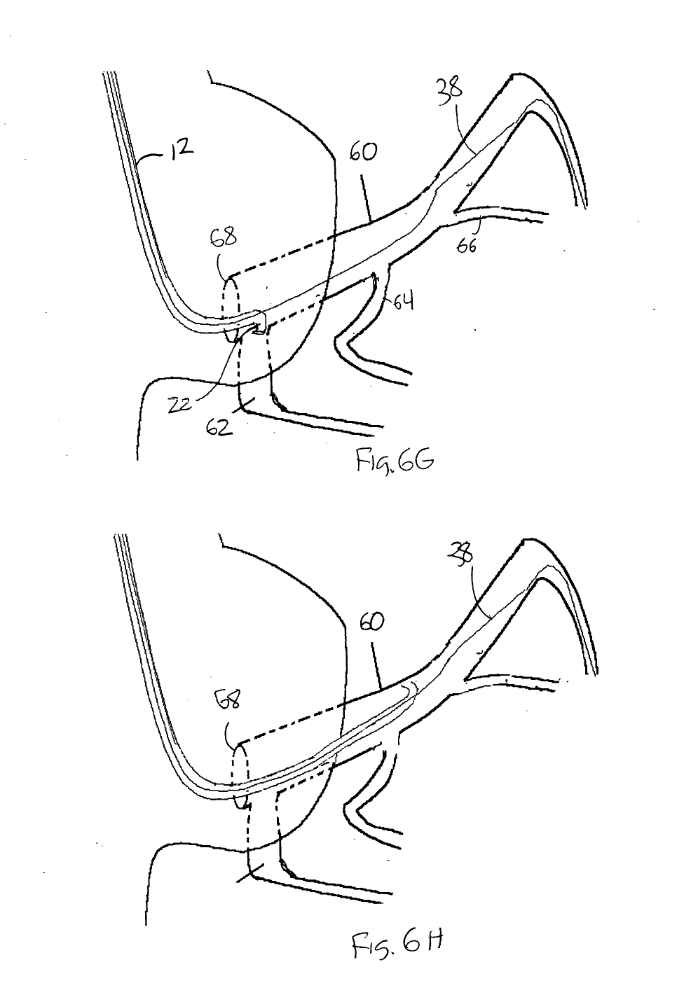

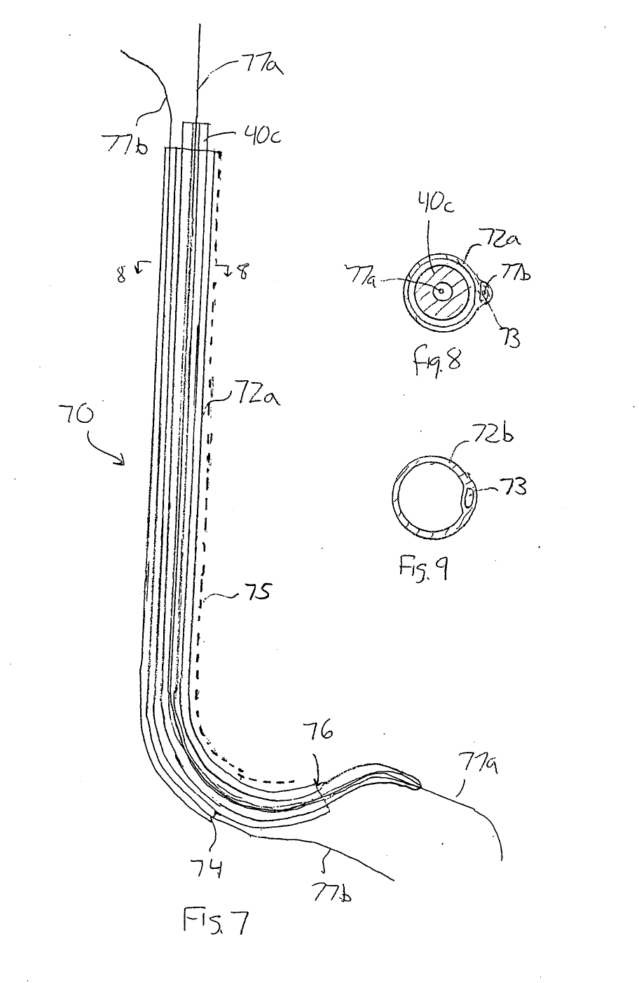

Referring to FIGS. 6A-6E, catheter system 10 may be used to cannulate a lateral branch of the coronary sinus as follows. Catheter 12 is advanced into the right atrium over guide wire 38 previously positioned through a venous sheath in the subclavian vein under fluoroscopy. Obturator 40a is advanced through catheter 12, and catheter system 10 is directed under fluoroscopy to the ostium 68 of the coronary sinus 60. Guide wire 38 is then advanced distally into the coronary sinus, after which catheter 12 and obturator 40a are advanced into the coronary sinus as illustrated in FIG. 6A. As shown, catheter system 10 with obturator 40a in catheter 12 is positioned in coronary sinus 60 and extends beyond first, second and third lateral branches 62, 64, 66 of coronary sinus 60.

In FIG. 6B, obturator 12 and guide wire 38 have been withdrawn, leaving catheter 12 in the coronary sinus with hooked shaped end 22 positioned beyond third lateral branch 66. A swan ganz catheter (not shown) is advanced through catheter 12 and angiography is preformed to identify the lateral branches. After the lateral branches have been identified, catheter 12 is gradually withdrawn. As catheter 12 is withdrawn, hooked shaped end 22 will engage lateral branches 66, 64 and 62 as it passes each branch, and this may eliminate the need to identify the locations of the lateral branches in advance. If a first or a subsequent branch engaged is not the target, catheter 12 is pulled past the branch and withdrawn until hook shaped end 22 engages the target branch. An electrical pacing lead may then be delivered through catheter 12 to the target branch.

This can be done with or without reintroduction of guide wire 38 to act as a support. In the embodiment shown, after positioning catheter 12 as shown in FIG. 6B, obturator 40c is advanced into catheter 12 until end opening 49 is positioned adjacent valve 30. Guide wire 38 is introduced through lumen 43 of obturator 40c and advanced distally into coronary sinus 60 as shown in FIG. 6C. Obturator 40c is then withdrawn, leaving guide wire 38 in position as shown in FIG. 6D. Catheter 12 may then be withdrawn along guide wire 38 to cannulate transverse branch 66 (FIG. 6E) or transverse branch 64 (FIG. 6F).

If the target branch is middle cardiac vein 62, additional care must be exercised. Due to the proximity of the middle cardiac vein 62 to ostium 68 of the coronary sinus 60, hook shaped end 22 of catheter 12 can inadvertently be completely withdrawn from the coronary sinus while attempting to cannulate vein 62. When middle cardiac vein 62 is the target, catheter 12 is withdrawn along guide wire 38 to cannulate middle cardiac vein 62 (FIG. 6G). If catheter 12 is withdrawn from coronary sinus 60 while attempting to cannulate middle cardiac vein 62, catheter 12 is re-introduced into coronary sinus 60 by advancing the catheter 12 along guide wire 38. If vein 62 proves to be inappropriate, another branch 64 or 66 can then be attempted by reintroducing catheter 12 along wire 38 as shown in FIG. 6H.

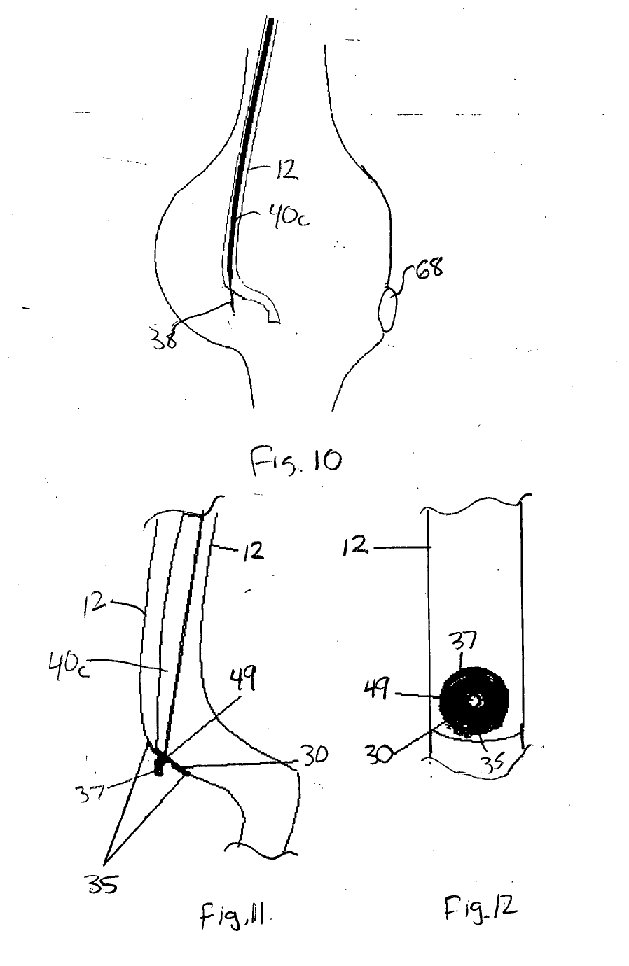

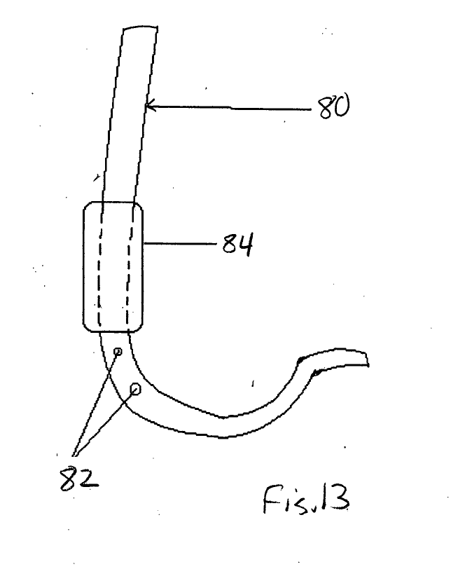

In an alternate embodiment of a catheter system 70 according to the invention shown in FIGS. 7 and 8, a catheter 72a is constructed with a side channel 73 formed as a rounded projection from its outer wall. In the alternative, side channel 73 can be formed in a catheter 72b substantially entirely inside the wall, causing only slight distortions of the catheter's round shape on its inner and outer diameters, as shown in FIG. 9. Side channel 73 terminates in an opening 74 at a location set back from the end opening 76 of catheter 72a near the distal bend therein at about the same location as valve 30 in the preceding embodiment. In this embodiment, a first guide wire 77a is extended from the obturator, e.g., 40c, and a second wire 77b which can be used as a stabilizing wire can be extended through channel 73 when called for in place of the wire extended through valve 30 in the procedure explained above. The side channel can also be relocated to the opposite side of the distal bend as shown in phantom lines 75, although this position is less convenient for positioning the stabilizing wire.

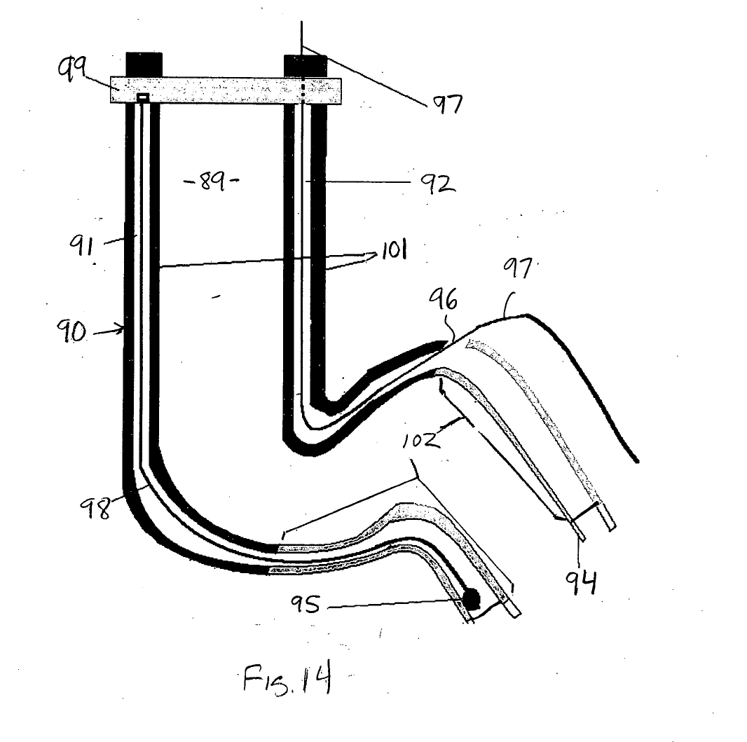

The foregoing procedures may be varied in a number of ways, with corresponding modifications to the structure of the catheter-obturator system. For example, as illustrated in FIGS. 10-12, a guide wire for use as a rail may be introduced in the right atrium before the catheter enters the coronary sinus. The stiff obturator 40c is introduced into catheter 12 until its tip 49 lies at silicone valve 30. Radiopaque markers 35, 37 define the location of silicone valve 30. Obturator 40c is advanced through valve 30. Then guide wire 38 is introduced into obturator 40c and allowed to exit its tip 49. Obturator 40c is withdrawn, leaving the guide wire 38 passed through silicone valve 30. Only about 0.5 cm of guide wire 38 is allowed to lie outside guide catheter 12. Shaped stylets can then be introduced into catheter 12, leaving the wire 38 in place. The coronary sinus is then cannulated, and guide wire 38 is advanced far into the coronary sinus to form a rail assuming the position of FIGS. 6E-6H.

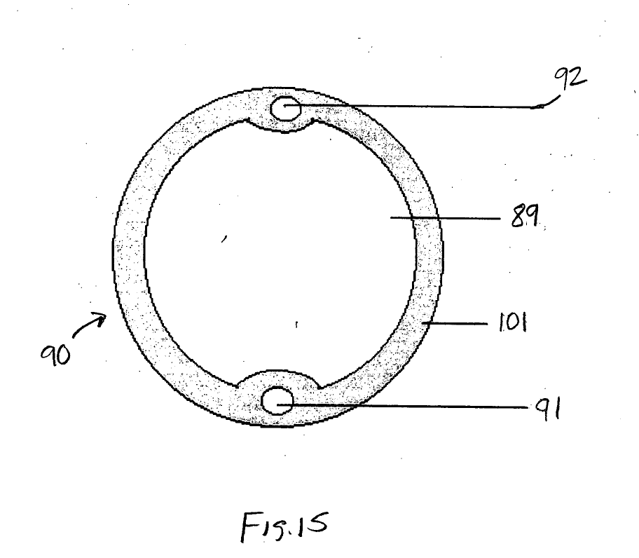

Referring to FIG. 13, for purposes of permitting the injection of contrast media in order to visualize the coronary sinus, the distal end (about 1 to 1.5 cm) of a modified catheter 80 of the invention may have from one to three spaced side holes 82 about 0.2-1 mm in diameter. These side holes 82 allow injection of contrast media to visualize the coronary sinus once the obturator is withdrawn and the curved tip of the guide catheter 80 is lying against the lateral wall of the coronary sinus. Catheter 80 also may have an inflatable balloon or cuff 84 thereon 2-4 cm from its tip, above holes 82. Balloon 84 is used to occlude the coronary sinus after air or contrast media is injected into it. This allows coronary sinus angiography without need for a swan ganz catheter as discussed above.

The size of balloon 84 may vary, but its diameter (inflated) is preferably 0.7-1.5 cm and its length is preferably 0.5-1.3 cm. Air may be introduced into balloon 84 via a channel in the wall of guide catheter 80. Contrast media can be introduced via the channel 73 for the stabilizing wire in the second embodiment discussed above, if channel 73 is provided with a silicone valve similar to valve 30 at its termination. In those embodiments that utilize a channel in the wall for a deflecting cable, described below, this channel may also be utilized for air injection into the balloon.

The catheter system of the invention can also be used in conjunction with the curvature adjusting mechanism described in Niazi U.S. Pat. No. 6,638,268, issued Oct. 28, 2003, the entire contents of which are hereby incorporated by reference herein. Specifically, the cable actuated system for changing the shape of the catheter can be applied to the catheter of the present invention. As shown schematically in FIGS. 14 and 15, a single deflecting catheter 90 has a pair of side channels 91, 92 formed in its sidewall at opposing positions. Channel 91 runs along the posterior wall of catheter 90 continuing to near the tip 94 of catheter 90. Channel 92 similarly runs to near tip 94, on the anterior side and has a slitted aperture 96 therein rearwardly spaced from tip 94 whereby a stabilizing wire 97 can be introduced into the coronary sinus and used in the manner described above for preceding embodiments. Channels 91, 92 are preferably on opposite sides of the lumen 89 of catheter 90. A metallic braided wire or nylon cord 98 runs through channel 91 and is attached at its termination to a fixation point 95 (e.g., weld) near tip 94. At its proximal end, cord 98 is attached to a proximal screw or ratchet mechanism 99. By turning the screw, or by retracting the ratchet, tension is exerted on the cord 98. This results in deflection of the guide catheter 90 such that its curvature is increased. By turning the screw in the opposite direction, or releasing the ratchet, tension is reduced in the cord 98, with relaxation of the curve.

This embodiment permits adjustment of the catheter shape while at the same time permitting movement along the coronary sinus using the stabilizing wire 97. In this embodiment, a stem portion 101 of catheter 90 is firm, a distal end portion 102 that starts near aperture 96 is soft, and the tip 94 is very soft. End portion 102 changes its curvature in response to tightening of ratchet mechanism 99 to a greater extent than stem portion 101.

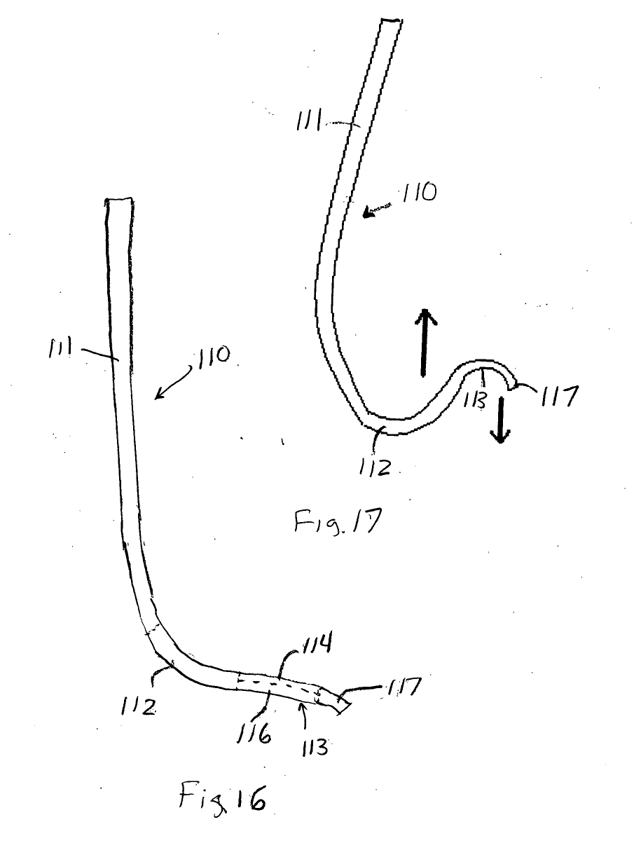

According to a further aspect of the invention, a dual curve deflectable guide catheter 110 as shown in FIGS. 16-18 is provided to cannulate coronary sinus branches. In this embodiment, the guide catheter 110 does not rely of shape memory of a hooked end as the means for entering the coronary sinus branches. Instead, catheter 110 has a tip which can be deflected upon actuation of a deflecting mechanism.

Referring to FIGS. 16-18, dual deflectable catheter 110 has a proximal end (stem or shaft) 111 which is firm and a secondary curve portion 112 which is softer, i.e., more pliable. The second segment or tertiary curve portion 113 after the secondary curve portion 112 is also soft to allow easy deflection. However, an anterior segment 114 of tertiary curve portion 113 is preferably of a firmer consistency than its posterior segment 116. This facilitates appropriate deflection of the tertiary curve 113 in a direction opposite to that of the primary curve. Second segment 113 ends in a short (e.g., 3 mm long) very soft, atraumatic tip 117 the purpose of which is to avoid dissection of the coronary sinus walls.

Two channels are present in the walls of the guide catheter 110. One channel 121 runs along the posterior wall of the guide, and ends at the termination of the tertiary curve near the tip 117. The second channel 122 runs along the anterior wall of the guide, and ends at the distal end of the catheter, just prior to tip 117. Metallic braided wires or nylon cords 123, 124 run through each channel 121, 122 and are attached at their terminations to fixation points 126, 127. Fixation point 126 is immediately proximate tip 117, whereas fixation point 127 is set back from point 126 along the length of catheter 110, at a position appropriate to cause bending of secondary curve 112.

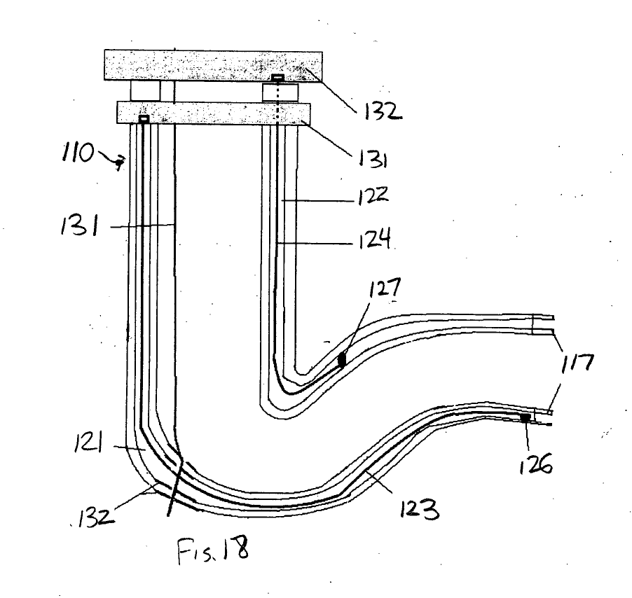

At their proximal ends, each cord 123, 124 is attached to respective proximal screw or ratchet mechanisms 131, 132. By turning the screw, or by retracting the ratchet, of mechanism 131, tension is exerted on the cord 123. This results in deflection of guide catheter 110 such that the curvature of the tertiary curve 113 is increased. Loosening of the cable 131 causes tertiary curve 113 to relax and catheter 110 returns to its undistorted position due to its resilient shape memory. Adjustment mechanism 132 operates in the same manner, but due to its point of attachment, acts to change the curvature of secondary curve 112. Stiff anterior segment 114 helps assure that tertiary curve will bend in the opposite direction to secondary curve 112, i.e. in the direction of the arrows as shown on FIG. 17, thereby producing a more S-shaped configuration suitable for navigating the coronary sinus.

Catheter 110 is introduced into the right atrium over a 0.035″ guide wire. It is rotated counterclockwise till it appears to point at the origin of the ostium of the coronary sinus in the fluoroscopic view. The guide wire is extended, and the coronary sinus ostium is probed for. Once the guide wire enters the coronary sinus, it is positioned as distally as possible in the coronary sinus system. The deflection of the guide catheter 110 at the secondary curve 112 helps to position the tip at the appropriate height, high above the floor of the right atrium for a high coronary sinus origin, lower for a lower origin. The tertiary curve 113 is kept at about 90 degrees in most cases, but may be increased or decreased if the coronary sinus is angulated sharply superiorly or inferiorly.

After cannulation, the tertiary curve 113 is relaxed till the tip 117 is almost parallel to the secondary curve 112. The secondary curve 112 is also relaxed, and the catheter 110 is advanced as far as possible into the coronary sinus system over the guide wire. The 0.035″ guide wire is then removed. Optionally, a stabilizing wire 131 may be passed through a distal silicone valve 132 using an obturator in the manner described above. The stabilizing wire 131 is positioned to act as a “rail” along which the catheter 110 can move as it is positioned or re-positioned in the coronary sinus.

Coronary sinus angiography is then performed. The tertiary curve 113 is increased to 60-120 degrees relative to its former near-parallel position, depending upon the angulation of the side branch to be cannulated. Catheter 110 is gently withdrawn, and with introduction of puffs of dye as visualization agent, the side branch orifice is then engaged. A 0.035″ wire is then advanced through catheter 110 into the target side branch. Over this wire, guide catheter 110 is selectively advanced into the side branch. The tertiary curve 113 is relaxed during this process.

A pacing lead is then delivered through catheter 110 into the target branch. If the location of the lead is considered suboptimal, catheter 110 is withdrawn into the main coronary sinus, the tertiary curve 113 is again increased, and catheter 110 withdrawn till a more proximal coronary sinus branch is cannulated selectively.

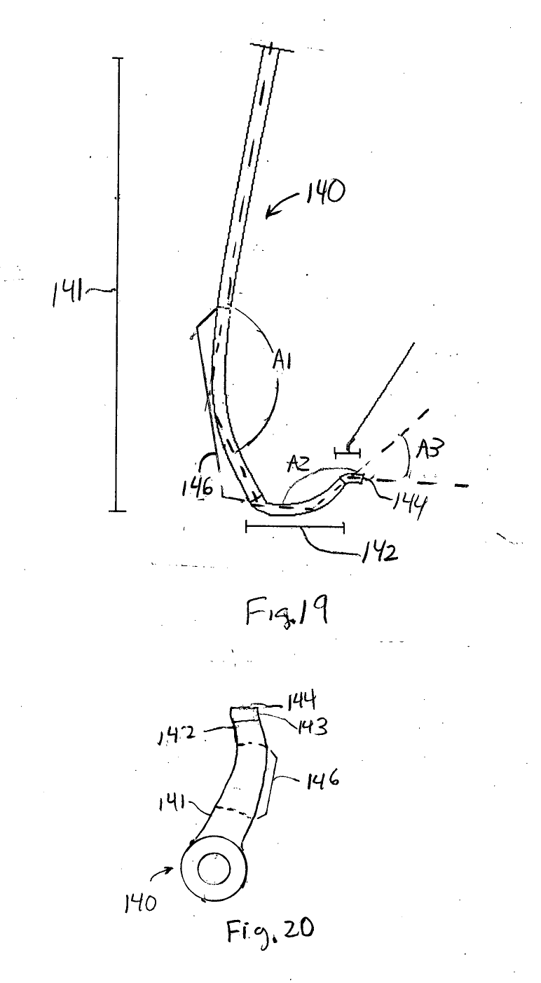

FIGS. 19 and 20 illustrate a further embodiment of a catheter 140 according to the invention. As in preceding embodiments, catheter has a primary curve 141, a secondary curve 142 and a tertiary curve 143 ending in a tip 144. Each of these curves generally lies in a common x-y plane. As a result, catheters 12, 80, 90 and 110 described above lie flat when in an undistorted condition. Catheter 140 additionally has a quaternary curve 146 which extends in the z-direction, to the left as shown in FIG. 20. This pre-curve is in a sideways direction when catheter 140 is held by the surgeon and makes it easier to locate the coronary sinus ostium when the catheter is in the right atrium. The overall angle A defined by the quaternary curve 146 is preferably in the range of about 10 to 45 degrees. The quaternary curve 146 is at an intermediate position along the length of the catheter and preferably overlaps one or both of the primary and secondary curves, giving catheter 140 a compound curvature at these locations.

Primary curve 141 is in the main shaft of catheter 140, and preferably varies at an angle A1 between about 180 (straight) and 145 degrees. Its main function is to adapt the catheter 140 to the curve of the superior vena cava and the right atrium. The secondary curve lies in the terminal 3 to 9 cm of catheter 140. Its size adapts the guide to right atria of various sizes, smaller sizes being appropriate for smaller atria and vice versa. The shape (angulation) of secondary curve 142 varies according to the height of the coronary sinus ostium above the right atrium floor. The greater the angulation, the higher the tip 144 lies above the right atrium floor. This angulation A2 varies from about 20 to 180 degrees. The tertiary curve 143 extends between the junction of the secondary curve 142 and tip 144. Its function is to direct the terminal part of catheter 140 in an appropriate direction for cannulation, either directly or with the help of a guide wire. Angle A3 varies between about 70 and 120 degrees. The foregoing angles are generally applicable to the earlier catheter embodiments 12, 80, 90, 110 as well as catheter 140. In each case, and the angles are measured as shown in FIG. 19 with reference to imaginary straight segments at opposite end of each curve but without regard for the extent of curvature in between, which may be continuous or contain a relatively sharp bend.

The quaternary curve 146 preferably lies mainly in the portion of the shaft of the obturator 140 that carries primary curve 141. Quaternary curve 146 preferably defines an angle greater than 0 but less than 90 degrees, preferably from about 10 to 45 degrees, out of the plane out of the plane of the other curves 141-143. Curve 146 adapts the guide to anatomy of the coronary sinus and right atrium. The length of obturator 140 is nominally 70 plus or minus 15 cm (55 cm-85 cm), with the last 5 to 10 cm extending out of the plane of the rest of the device. The length of the soft tip segment of the guide catheter 140 is preferably 0.3 (30%) of the diameter of the guide catheter, which diameter may vary about 7 to 10 French.

The claims which follow define certain aspects of the invention but do not limit the invention. For example, a catheter system according to the invention could be used to reach other hard to access parts of the human body due to its unique configuration.

1. A catheter system suitable for introduction of a pacing lead into a branch of a human coronary sinus, comprising:
a resilient catheter having shape memory and which is configured for introduction into the coronary sinus from the right atrium of a human heart, the catheter having at least one preformed bend defining an acute included angle proximate a tip thereof, such that the catheter has a hooked end portion when in an undistorted condition; and
a stiff obturator configured for sliding into and out of the catheter, wherein a distal end portion of the obturator has a substantially straight configuration whereby introduction of the obturator fully into the catheter straightens the hooked end portion, and withdrawal of the obturator causes the hooked end portion to resume its hooked shape.

2. The catheter system of claim 1, further comprising means for extending a guide wire extended through the catheter out of the catheter from a location above the hooked end.

3. The catheter system of claim 2, wherein the means for extending a guide wire comprises a valve formed in a side wall of the catheter, whereby a guide wire can be introduced through the catheter and valve into the coronary sinus.

4. The catheter system of claim 2, wherein the means for extending a guide wire comprises a longitudinal channel formed in a side wall of the catheter.

5. The catheter system of claim 1 wherein the catheter is substantially J-shaped in an undistorted condition.

6. The catheter system of claim 1 wherein the obturator is configured for insertion into the catheter when the catheter is disposed in a coronary sinus.

7. The catheter system of claim 5, wherein a stem portion of the catheter is substantially straight, a curved mid-portion of the catheter is thinner and less stiff than the stem portion, and the hooked end portion, which extends from the mid-portion, is thinner and less stiff than the mid-portion.

8. A catheter system suitable for introduction of a pacing lead into a branch of a human coronary sinus, comprising:
a resilient catheter having shape memory and which is configured for introduction into the coronary sinus from the right atrium of a human heart, the catheter having at least one preformed bend;
a mechanism operable from the proximal end of the catheter for changing the curvature of a distal end portion of the catheter, including a cable anchored to the catheter at a point proximate the distal end of the catheter and at a point near the proximal end of the catheter, whereby shortening of the cable from the point near the proximal end of the catheter results in increased curvature of the distal end portion; and
means for extending a guide wire extended through the catheter out of the catheter from a location above the distal end portion.

9. The catheter system of claim 8, wherein the mechanism for changing the curvature includes a hand-rotatable screw on which the cable is wound at the proximate end of the catheter.

10. The catheter system of claim 9, wherein the cable is mounted in a channel in a wall of the catheter.

11. The catheter system of claim 8, wherein the means for extending a guide wire comprises a valve formed in a side wall of the catheter, whereby a guide wire can be introduced through the catheter and valve into the coronary sinus.

12. A catheter system suitable for introduction of a pacing lead into a branch of a human coronary sinus, comprising:
a resilient catheter having shape memory and which is configured for introduction into the coronary sinus from the right atrium of a human heart, the catheter having at least one preformed bend;
a first mechanism operable from the proximal end of the catheter for changing the curvature of a distal end portion of the catheter, including a first cable anchored to the catheter at a point proximate the distal end of the catheter and at a point near the proximal end of the catheter, whereby shortening of the first cable from the point near the proximal end of the catheter results in increased curvature of the distal end portion; and
a second mechanism operable from the proximal end of the catheter for changing the curvature of the distal end of the catheter, including a second cable anchored to the catheter at a point near the distal end of the catheter different from the point at which the first cable is attached, and at a point proximate the proximal end of the catheter, whereby shortening of the second cable from the point near the proximal end of the catheter results in increased curvature of the distal end portion at a location different from the increased curvature resulting from shortening of the cable of the first mechanism.

13. The catheter system of claim 12, wherein the first cable extends through a first channel formed in a wall of the catheter on its posterior side, and the second cable extends through a second channel formed in a wall of the catheter on its anterior side.

14. The catheter system of claim 13, wherein the first and second channels are in opposing positions.

15. The catheter system of claim 12, wherein the first cable is anchored at a point adjacent the tip of the catheter and the second cable is anchored at a point set back from the tip of the catheter, such that shortening of the first cable increases curvature along a first segment proximate the tip, and shortening of the second cable increases curvature along a second segment set back from the first segment.

16. The catheter system of claim 15, wherein the first and second segments curve in opposite directions, such that shortening of the first and second cables causes a distal end portion of the catheter to assume an S-shaped configuration.

17. The catheter system of claim 15, further comprising means for extending a guide wire extended through the catheter out of the catheter from a location above the distal end portion.

18. A resilient catheter having shape memory which is configured for introduction into the coronary sinus from the right atrium of a human heart, the catheter having at least two first and second preformed bends giving the catheter a curved, hooked shape proximate a tip thereof, which first and second bends lies in a common plane, and a third preformed bend defining an acute included angle, which third bend extends out of the common plane of the first and second bends.

19. The catheter of claim 18, wherein the first and second bends render the catheter substantially J-shaped when in an undistorted condition, and the third preformed bend coincides with the first bend and pre-bends the catheter to the left when the catheter is viewed from its proximal end.

20. The catheter of claim 19, wherein the third preformed bend defines an angle in the range of about 10 to 45 degrees.
